(19) United States
(12) Patent Application Publication
(10) Pub. No.: US 2017/0188974 A1
(43) Pub. Date: Jul. 6, 2017

(54) Evaluating multivariate response of circadian rhythms

(71) Applicant: Qingbo Li, Bolingbrook, IL (US)

(72) Inventor: Qingbo Li, Bolingbrook, IL (US)

(21) Appl. No.: 15/115,690

(22) PCT Filed: Feb. 3, 2015
(86) PCT No.: PCT/US2015/014155

§ 371 Date: Aug. 1, 2016

Related U.S. Application Data
(60) Provisional application No. 61/934,870, filed on Feb. 3, 2014.
(60) Provisional application No. 61/947,785, filed on Mar. 4, 2014.

(51) Int. Cl. A61B 5/00; A61B 5/145; A61B 5/0205

(52) U.S. Cl. CPC A61B 5/7275; A61B 5/4857; A61B 5/0205; A61B 5/145

(57) ABSTRACT

A statistical system is disclosed to analyze multivariate response of circadian rhythms in a crossover design, in which response variables are continuously monitored to evaluate the therapeutic effect of a regimen on circadian rhythms such as blood pressure and blood sugar. The methods determine the alteration of not only amplitude but also correlation of multiple circadian rhythms under the influence of a regimen.

### 1. FIELD OF INVENTION

The present invention relates to statistical methods for effectively assessing how a regimen might affect multiple circadian rhythms, for example blood pressure and blood sugar, jointly.

### 2. BACKGROUND OF THE INVENTION

Diabetes and hypertension are two conditions that often coexist. Their combination increases the risk of life-threatening cardiovascular events. High blood pressure contributes to several of the classic diabetic complications, and high blood sugars cause high blood pressure. Most subjects with diabetics require a combination therapy to control blood pressure to a goal of <130/80 mmHg. The potential for some drugs to reduce blood pressure, however, might be offset by an increased risk due to the development of diabetics. Controlling high blood pressure may be as important as controlling our blood sugar. There have been ample examples of medicines that could act on both blood pressure and blood sugar in the same or opposite way, intentionally or unintentionally. Thus, simultaneous monitoring of blood pressure and sugar level will be informative in preventive management and therapy for either or both of the two disease conditions.

Both blood pressure and blood sugar exhibit apparent circadian rhythm. The circadian patterns often make it difficult to choose a single time-point marker for therapeutic and preventive management. For example, the daytime systolic blood pressure variability is a stronger predictor of early carotid atherosclerosis progression than a single time-point reading. It is useful to define the risk-benefit ratio of therapeutic approaches.

Several manufacturers have produced medical devices to continuously monitor blood pressure and blood sugar. Some of these devices transmit the readings via wireless technologies to portable devices or network servers for analysis. Some of these devices have been cleared by FDA. For example, Sotera Wireless has been granted FDA approval for the ViSi Mobile continuous, non-invasive blood pressure (cNIBP) monitoring. Dexcom G4 Platinum Continuous Glucose Monitor was approved by FDA. These devices provide rich data that can be used for more precise evaluation of therapies or for better preventive health management.

Computationally stable and efficient statistical methods to make full use of the rich data provided by these continuous monitoring devices, however, are still needed. With an increased number of data points and repeated measurements, the number of parameters in the variance-covariance matrices increases accordingly. While an overly simplified variance-covariance structure introduces biases, a totally unstructured variance-covariance matrix for a model introduces such a large number of components that become impracticable to estimate. Thus, a balance needs to reach among minimizing bias, increasing efficiency, and reducing the number of variance-covariance components to estimate. This disclosure aims to provide statistical methods to achieve such a goal based on a crossover design.

With increasing capacity of upcoming devices to record circadian rhythms at higher sampling frequency, it may sometimes be desirable to use derived statistics of those readings for statistical modeling and analysis. Within such a context, readings or measurements can mean either the original data outputs or their summary statistics suitable to describe circadian rhythms such as amplitude, acrophase, mesor, mean, median, variability, variance, standard deviation, range, or selected subset of data points, etc.

Portable and wireless devices have dramatically changed how medical data are collected, transmitted, and stored. While many inventions and products have shown or provide ways to record multiple vital signs through different sensors that are bundled together, perhaps not enough emphasis has been made on compact and integrated devices that provide precisely synchronized measurements of vital signs especially blood pressure and glucose concentration. Precisely synchronized device typically provides more reliable time-matched measurements and more reliable data analysis. Because many blood pressure and blood sugar monitoring devices are usually based on very different sensing techniques, precise synchronization in data collection for both circadian rhythms are not a trivial issue.

Although the disclosure is illustrated for responses in a circadian rhythm, it is also suitable for other cyclic rhythms. For example, the circadian rhythm can be replaced by a monthly cycle, and the day can be accordingly replaced by the month. Such an adaptation can be implemented without having to change the statistical model and methods established for the circadian system.

Although blood pressure and blood glucose level are used to illustrate the statistical model in this disclosure, the response variables do not have to be different types biological or chemical signals as distinguished as blood pressure and blood glucose. They can be the same biological signal, such as blood pressure or blood glucose, respectively, measured by medical devices from different manufacturers. In that scenario, the purpose of the study may include an evaluation of the equivalence of two devices to measure the same signal. In another scenario, one of the response variables may simply be a baseline reading of a response variable preceding a period in a sequence. The inclusion of a baseline as a response in a linear mixed statistical model could improve statistical efficiency a crossover design.

### 3. OBJECTS OF THE INVENTION

It is an object of this invention to use joint modeling of multivariate response and crossover design to efficiently determine the effect of a regimen on multiple circadian rhythms with minimum sample sizes. The utility of the invention has the advantages to determine not only the change of amplitude but also the alteration of correlation for different circadian rhythms under treatment of a regimen. In other words, the methods provide a precise way to assess whether a regimen acts on multiple circadian rhythms jointly or independently.

### 4. SUMMARY OF THE INVENTION

A statistical system is disclosed to analyze multivariate response of circadian rhythms in a crossover design, in which response variables are continuously or discretely monitored to provide multiple-time-point data for an evaluation of a therapeutic effect of a regimen. Multiple readings are taken to account for the circadian rhythm of a study subject. Readings can be the original data outputs or summary statistics of the outputs suitable to describe circadian rhythms such as amplitude, acrophase, mesor, mean, median, variability, variance, standard deviation, range, or selected subset of data points, etc.

The response variables, which can represent all readout data points or their statistics, are monitored in a synchronized fashion so that their paired readings are taken at the same time-point or in a time window, which is within one hour, preferably within fifteen minutes, more preferably within five minutes, and most preferably within 30 seconds. One integrated receiver assembly receives readings from sensors that measure the response variables. The integrated receiver assembly contains one antenna or multiple antennas that space not further apart than a distance limit, which is within eight inches, preferably within four inches, more preferably within two inches, and most preferably within one inch. The integrated receiver assembly is housed in an enclosure or on a mounting base not to exceed a weight limit, which is within ten ounces, preferably within seven ounces, more preferably within four ounces, and most preferably within two ounces. The enclosure or mounting base is constructed from polymer, fabric, glass, metal, plastic, alloy, wood, paper or a combination of these materials. The receiver receives, stores, and transfers the readings to a computing device that implements a statistical model to analyze the data.

In one embodiment of the statistical model, a sequence in a crossover design consists of multiple periods. Each period consists of multiple days. Measurements are made each day at multiple time-points. At each time-point, multiple response variables are monitored. The response variables are potentially correlated at each time-point, between time-points on a day, between days in a period, and between periods in a sequence. Using a unified variance-covariance structure, the disclosed method limits the number of parameters that need to be estimated for a variance-covariance matrix. The number of parameters to estimate for the variance-covariance matrix of the response variables remains the same even when the numbers of time-points, days, and periods increase. Limiting the number of parameters to estimate for a variance-covariance matrix improves statistical efficiency and inference precision. The method is suitable for making a full use of highly repeated measurements for a circadian system. Making a full use of highly repeated measurements for a circadian system improves the precision to estimate the correlation of the response variables. The estimated change in the correlation of the response variables assists one to determine whether a regimen induces an independent response in one response variable or correlated changes between response variables. The objects, features and advantages of this disclosure will be illustrated in more details in the following description of the embodiments, the examples, and the appended claims.

### 5. BRIEF DESCRIPTION OF FIGURES

FIG. 1 illustrates the joint response of blood pressure and blood glucose to two regimens with insignificant difference in efficacy in a 2×2 crossover design.

FIG. 2 illustrates the joint response of blood pressure and blood glucose to two regimens with significant difference in efficacy in a 2×2 crossover design.

FIG. 3 illustrates the response of blood pressure to two regimens relative to baseline in a 2×2 crossover design.

### 6. DETAILED DESCRIPTION OF THE PREFERRED EMBODIMENTS

### Example 1: Simulated Joint Response of Blood Pressure and Blood Sugar to Two Regimens with Negligible Difference in Efficacy

It is well known that a crossover design has the advantage of minimizing between-subject variation thus requires a smaller number of subjects for efficient statistical analysis. In this example, a 2×2 crossover design is set up with three days in each period. Each day has five measurements.

Let {X, Y} be the vector of the bi-variate responses of blood pressure X and blood sugar Y. The variance-covariance matrix Σ for the response vector {X, Y} can be described as following at different levels that include between-period

$${\sum }_{Bp} = ( \begin{matrix}{ζ}_{xx} & {ζ}_{xy} \\ {ζ}_{xy} & {ζ}_{yy}\end{matrix} ) ,$$

between-day

$${\sum }_{Bd} = ( \begin{matrix}{η}_{xx} & {η}_{xy} \\ {η}_{xy} & {η}_{yy}\end{matrix} ) ,$$

between-timepoint

$${\sum }_{Bt} = ( \begin{matrix}{υ}_{xx} & {υ}_{xy} \\ {υ}_{xy} & {υ}_{yy}\end{matrix} ) ,$$

and within-timepoint

$${\sum }_{Wt} = ( \begin{matrix}{σ}_{xx} & {σ}_{xy} \\ {σ}_{xy} & {σ}_{yy}\end{matrix} ) .$$

For the 2×2×3×5 response vector (Xᵢ₁₁₁ₖ, Yᵢ₁₁₁ₖ, Xᵢ₁₁₂ₖ, Yᵢ₁₁₂ₖ, . . . , Xᵢ₂₃₅ₖ, Y₂₃₅ₖ) of the k^{th} subject in the i^{th} sequence, the variance-covariance structure can be constructed based on a compound symmetry structure, as shown in the following.

For a day, the 2×5 response vector (X_{icb1k}, Y_{icb1k}, X_{icb2k}, Y_{icb2k}, . . . , X_{icb5k}, Y_{icb5k}) of the k^{th} subject in the i^{th} sequence in the c^{th} period on the b^{th} day has the following variance-covariance matrix Covd=Σ_{Wt} [symbol] I(t)+E_{Bt} [symbol] J (t, t) where I is an identity matrix and J is a matrix of ones.

For a period, the 2×3×5 response vector (X_{ic11k}, Y_{ic11k}, X_{ic12k}, Y_{ic12k}, . . . , Y_{ic35k}, Y_{ic35k}) of the k^{th} subject in the i^{th} sequence in the c^{th} period has the following variance-covariance matrix Covp=Covd [symbol] I(t×d)+E_{Bd} [symbol] J(t×d,t×d).

For a sequence, the 2×2×3×5 response vector (Xᵢ₁₁₁ₖ Yᵢ₁₁₁ₖ, Xᵢ₁₁₂ₖ, Yᵢ₁₁₂ₖ, . . . , Xᵢ₂₃₅ₖ, Y₂₃₅ₖ) of the k^{th} subject in the i^{th} sequence has the following variance-covariance matrix Cov=Covp [symbol] I(t×d×p)+E_{Bp} [symbol] J(t×d×p,t×d×p).

The statistical model can be implemented as a mixed model in statistical software SAS. Let Z={X, Y}. Then the mixed model can be expressed as Model Z=Period*Response Period*Day*Response Period*Day*Time*Response Day*Time*Regimen*Response; random Response/subject=Subject Type=UN; random Response/subject=Subject*Period Type=UN; random Response/Subject=Subject*Period*Day Type=UN; repeated Response/Subject=Subject*Period *Day*Time Type=UN; LSMEANS Day*Time*Regimen*Response/PDIFF; in which Subject is a study subject in the i^{th} sequence, Period is the period index taking a value between 1 and p=2, Day is the day index taking a value between 1 and d=3, Time is the time-point index taking a value between 1 and t=5, Regimen is the regimen index taking a value from 1 up to p=2, Response is the type index of a response variable with 1 for blood pressure and 2 for blood sugar. Finally, Z is the 2×2×3×5 response vector (Xᵢ₁₁₁ₖ, Yᵢ₁₁₁ₖ, Yᵢ₁₁₂ₖ, Yᵢ₁₁₂ₖ, . . . , Xᵢ₂₃₅ₖ, Yᵢ₂₃₅ₖ).

The variance-covariance matrix Cov for the 2×2×3×5 response vector (Xᵢ₁₁₁ₖ, Yᵢ₁₁₁ₖ, Xᵢ₁₁₂ₖ, Yᵢ₁₁₂ₖ, . . . , Xᵢ₂₃₅ₖ, Yᵢ₂₃₅ₖ) was simulated with the following parameters and SAS expressions. Let Wt={12 3, 3 16}, Bt={30 8, 8 32}, Bd={52-12, -12 56}, and Bp={59 64, 64 101}. Then WtI=I(t)@Wt, WdJ=J(t,t)@Bt, Covd=WtI+WdJ, WdI=I(d)@Covd, WpJ=J(t*d,t*d)@Bd, Covp=WdI+WpJ, WpI=I(p) @Covp, BJ=J(t*d*p,t*d*p)@Bp. Eventually, Cov=WpI+BJ.

The sample response values for Z={X, Y} were then generated by using the SAS function as RANDNORMAL(1, E(Zᵢₖ), Cov), where E(Zᵢₖ) was the expected values of the response vector Z={X, Y} and Cov as computed above.

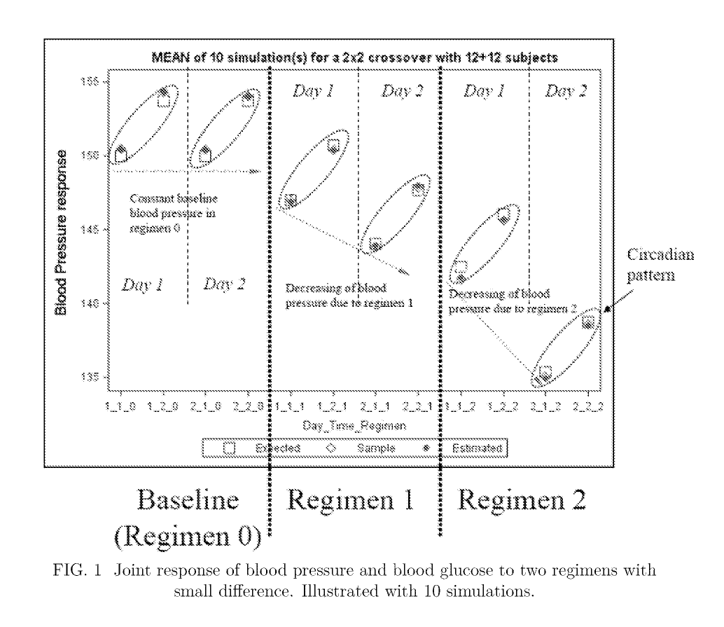

A set of simulation results are presented in FIG. 1. Blood pressure and blood glucose responses were simulated 10 times based on the above mentioned mixed model in a 2×2 crossover design with 9 subjects in each sequence. The responses were the average of the 10 simulations. The graph is divided into quadrants based on two response variables and two regimens as labeled. Vertically, the symbols in the top half are the blood pressure values, and those in the bottom half are the blood glucose values. Horizontally, the left half represents the responses in regimen 1, and the other half to the right represents the responses due to regimen 2. The circadian patterns of both responses are indicated with dashed eclipses to the right side of the graph. In each regimen, the response values are sorted by the order of Day and Time-point. The daily changes in responses due to the regimen effects are indicated with the dashed arrows. At each time-point, a response includes its expected value (□), its sample mean (⋄), and its estimated value from the statistical model (). In this simulation study, the two regimen effects were set to be equivalent. In each regimen, the responses gradually decreased over a 5-day period while the circadian pattern remained the same through out the days. The expected value, sample mean, and estimated value agreed with each other at the timepoints.

### Example 2: Simulated Joint Response of Blood Pressure and Blood Sugar to Two Regimens with Significant Difference in Efficacy

This example has variance-covariance matrices similar to those in the last example except that the two regimens have significant difference in efficacy. Blood pressure and blood glucose responses were simulated 1 time based on the above described mixed model in a 2×2 crossover design with 9 subjects in each sequence. The graph is divided into quadrants based on two response variables and two regimens as labeled. Vertically, the symbols in the top half are the blood pressure values, and those in the bottom half are the blood glucose values. Horizontally, the left half represents the responses in regimen 1, and the other half to the right represents the responses due to regimen 2. The circadian patterns of both responses are indicated with dashed eclipses to the right side of the graph. In each regimen, the response values are sorted by the order of Day and Time-point. The daily changes in responses due to the regimen effects are indicated with the dashed arrows. At each timepoint, a response includes its expected value (□), its sample mean (⋄), and its estimated value from the statistical model (). In this simulation study, the two regimen effects were set to be different with regimen 2 having a larger effect in decreasing the responses. In each regimen, the responses gradually decreased over a 5-day period while the circadian pattern remained similar through out the days. Even with only one simulation, the expected value, sample mean, and estimated value reasonably agreed with each other at every time-point. The sample size of 18 used in the study is within the typical range of sample sizes in a 2×2 crossover design where 18-24 subjects are usually used.

### Example 3: Simulated Response of Blood Pressure to Two Regimens Relative to Baseline in a 2×2 Crossover Design

A baseline typically means a reading of a vital sign when a subject does not receive any treatment. Under certain circumstances where a difference between baseline and placebo is difficult to distinguish, it also means a reading of a vital sign when a subject receives a blank treatment, or no treatment at all. For example, when a normal meal and a specially design meal are to be compared, the normal meal serves as placebo as well as baseline. In another example when alcohol consumption is compared with non-alcohol consumption, a placebo is also difficult to define. In such a case, non-alcohol consumption is treated as the norm, the baseline, and the placebo. Other examples include high-salt versus low-salt, high-fat versus low-fat, high-sugar versus low-sugar, etc. In these situations, an estimation of the difference between baseline and a regimen has its meaning in evaluation of a therapeutic effect.

When the effect of a regimen is evaluated against an upper safety threshold, for example, the highest blood pressure of a day that should not be exceeded, a placebo effect may not need to be subtracted from a therapeutic effect if it is confounded with a placebo effect. In such a case, the estimate of a change from baseline for a regimen serves as a more conservative safety gauge than an estimate of the difference between a regimen and aZ={X, Y} represents a placebo, even when the placebo can be distinguished from the baseline.

This example illustrates a 2×2 crossover design with two days in each period and two measurements each day in the layout, which models blood pressure with baseline as joint response. The settings are similar to the above examples except that Z={X, Y} represents a vector with X denoting the baseline blood pressure prior to treatment and Y denoting the blood pressure response to a regimen after the baseline.

Similar to the above examples, the variance-covariance matrix Σ for the response vector {X, Y} can be described as following at different levels that include between-period

$${\sum }_{Bp} = ( \begin{matrix}{ζ}_{xx} & {ζ}_{xy} \\ {ζ}_{xy} & {ζ}_{yy}\end{matrix} ) ,$$

between-day

$${\sum }_{Bd} = ( \begin{matrix}{η}_{xx} & {η}_{xy} \\ {η}_{xy} & {η}_{yy}\end{matrix} ) ,$$

between-timepoint

$${\sum }_{Bt} = ( \begin{matrix}{υ}_{xx} & {υ}_{xy} \\ {υ}_{xy} & {υ}_{yy}\end{matrix} ) ,$$

and within-timepoint

$${\sum }_{Wt} = ( \begin{matrix}{σ}_{xx} & {σ}_{xy} \\ {σ}_{xy} & {σ}_{yy}\end{matrix} ) .$$

For the 2×2×2×2 response vector (Xᵢ₁₁₁ₖ, Yᵢ₁₁₁ₖ, Xᵢ₁₁₂ₖ, Yᵢ₁₁₂ₖ, . . . , Xᵢ₂₂₂ₖ, Yᵢ₂₂₂ₖ) of the k^{th} subject in the i^{th} sequence, the variance-covariance structure can be constructed based on a compound symmetry structure, as shown in the following.

For a day, the 2×2 response vector (X_{icb1k}, Y_{icb1k}, X_{icb2k}, Y_{icb2k}) of the k^{th} subject in the i^{th} sequence in the c^{th} period on the b^{th} day has the following variance-covariance matrix Covd=Σ_{wt} [symbol] I(t)+Σ_{Bt} [symbol] (t, t) where I is an identity matrix and J is a matrix of ones.

For a period, the 2×2×2 response vector (X_{ic11k}, Y_{ic11k}, X_{ic12k}, Y_{ic12k}, X_{ic21k}, Y_{ic21k}, X_{ic22k}, Y_{ic22k}) of the k^{th} subject in the i^{th} sequence in the c^{th} period has the following variance-covariance matrix Covp=Covd [symbol] I(t×d)+E_{Bd} [symbol] (t×d,t×d).

For a sequence, the 2×2×2×2 response vector (Xᵢ₁₁₁ₖ, Yᵢ₁₁₁ₖ, Xᵢ₁₁₂ₖ, Yᵢ₁₁₂ₖ, . . . , Xᵢ₂₂₂ₖ, Yᵢ₂₂₂ₖ) of the k^{th} subject in the i^{th} sequence has the following variance-covariance matrix Cov=Covp [symbol] I(t×d×p)+Σ_{Bp} [symbol] J(t×d×p,t×d×p).

The statistical model can be implemented as a mixed model in statistical software SAS. Let Z={X, Y}. Then the mixed model can be expressed as Model Z=Period*Response Period*Day*Response Period*Day*Time*Response Day*Time*Regimen*Response; random Response/subject=Subject Type=UN; random Response/subject=Subject*Period Type=UN; random Response/Subject=Subject*Period*Day Type=UN; repeated Response/Subject=Subject*Period *Day*Time Type=UN; LSMEANS Day*Time*Regimen*Response/PDIFF; in which Subject is a study subject in the i^{th} sequence, Period is the period index taking a value of 1 or 2, Day is the day index taking a value of 1 or 2, Time is the time-point index taking a value of 1 or 2, Regimen is the regimen index taking a value of 0, 1, or 2, Response is the type index of a response variable with 0 for baseline blood pressure and 1 for blood pressure response. Finally, Z is the 2×2×2×2 response vector (Xᵢ₁₁₁ₖ, Yᵢ₁₁₁ₖ, Xᵢ₁₁₂ₖ, Yᵢ₁₁₂ₖ, . . . , Xᵢ₂₂₂ₖ, Yᵢ₂₂₂ₖ).

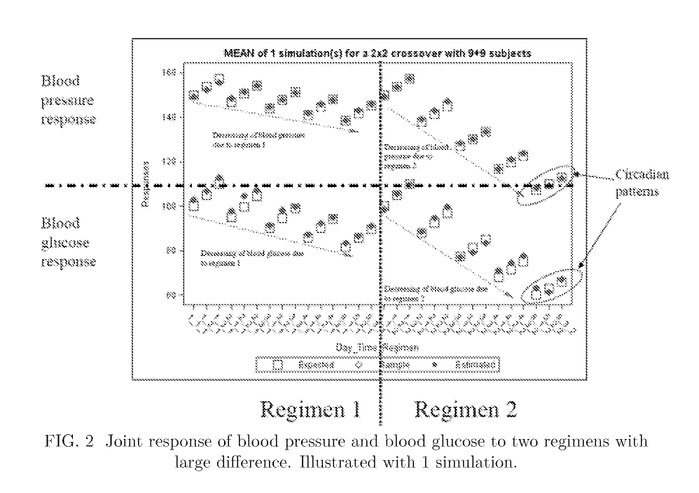

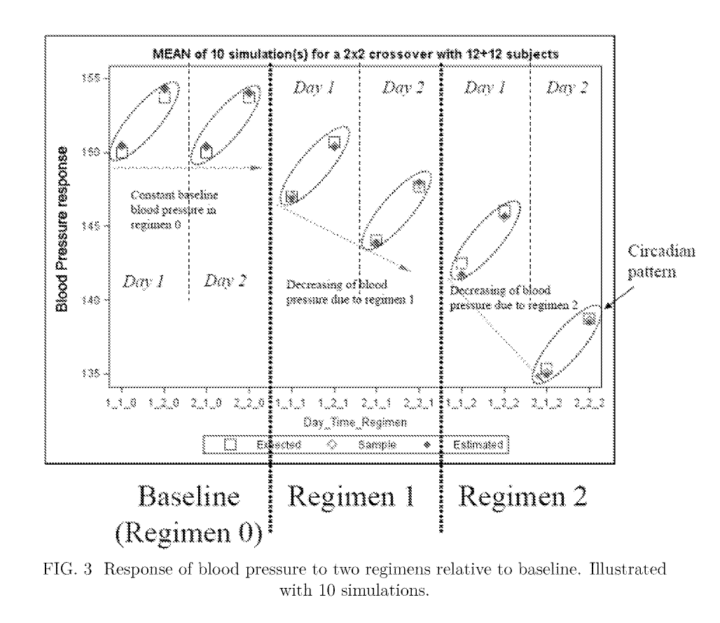

A set of simulation results are illustrated in FIG. 3. The responses were the average of 10 simulations. Legends are similar to those in FIG. 1 and FIG. 2. The blood pressure responses decrease in regimens 1 and 2 due to the regimen effects compared to baseline (regimen 0). Regimen 2 has a larger effect than regimen 1.

SYNCHRONIZED DATA COLLECTION: To achieve reliable data collection and precise data analysis in a joint modeling, response variables for circadian rhythms are to be monitored in a synchronized fashion so that their paired readings are taken at the same time-point or in a time window, which is within one hour, preferably within fifteen minutes, more preferably within five minutes, and most preferably within 30 seconds.

Synchronization of data collection are to be proactively programmed in an integrated receiver assembly receives readings from sensors that measure the response variables. The integrated receiver assembly contains one antenna or multiple antennas that space not further apart than a distance limit, which is within eight inches, preferably within four inches, more preferably within two inches, and most preferably within one inch. Here, an antenna refers to a general type of data signal entry point that is implemented with either a wireless technique or a wired technique in which a conduit is used to physically connect a sensor and the receiver. The integrated receiver assembly is housed in an enclosure or on a mounting base not to exceed a weight limit, which is within ten ounces, preferably within seven ounces, more preferably within four ounces, and most preferably within two ounces. The enclosure or mounting base is constructed from polymer, fabric, glass, metal, plastic, alloy, wood, paper or a combination of these materials. The receiver receives, stores, and transfers the readings to a computing device that implements a statistical model to analyze the data. The computing device is either housed in the same enclosure with the receiver or separated from the receiver. The readings may be log-transformed or subjected to other normalization prior to statistical analysis.

### REFERENCES

[1] ForaCare. Blood Glucose Plus Blood Pressure Monitoring System D40 (www.foracare.com).
[2] B. M. Alman. Automated patient monitoring and counseling system [US20070106127], 2007.
[3] M. Ballet, et. al. Wireless, internet-based medical-diagnostic system [U.S. Pat. No. 7,396,330], 2008.
[4] D. Blackburn and T Wilson. Antihypertensive medications and blood sugar: Theories and implications. Can J Cardiol, 22(3):229-233, 2006.
[5] N. Brown. Cardiovascular Effects of Anti-Diabetic Agents: Focus on Blood Pressure Effects of Incretin-Based Therapies. J Am Soc Hypertens, 6(3):163-168, 2012.
[6] D. Doreus and E. Doreus. All in one medical monitor [US20120041276], 2012.
[7] Enric Enric Monte Moreno. System and method for the simultaneous, non-invasive estimation of blood glucose, glucocorticoid level and blood pressure [US20130267796], 2013.
[8] A. Fontana, et. al. A linear mixed model approach to compare the evolution of multiple biological rhythms. Statistics in medicine, 32(7):1125-35, 2013.
[9] H. C. Gerstein and S. Yusuf. Clinical outcomes trials and the cardiovascular effects of thiazolidinediones: implications for the evaluation of anti-diabetic drugs. American heart journal, 160(1):1-2, 2010.
[10] G. Hayter, et. al. Method and apparatus for providing data processing and control in a medical communication system [U.S. Pat. No. 8,600,681], 2013.
[11] M. C. Jemison, et. al. Real-time and simultaneous monitoring of multiple parameters from multiple living beings [EP1703838A2], 2006.
[12] R. Y. Jiff and M. K. Sloan. Continuous glucose monitoring system and methods of use [U.S. Pat. No. 8,622,903], 2014.
[13] S. S. Khanuja, et. al. Method and apparatus for remotely monitoring the condition of a patient [U.S. Pat. No. 7,448,996], 2008.
[14] M. G. Kenward and J. H. Roger. The use of baseline covariates in crossover studies. Biostatistics (Oxford, England), 11(1):1-17, 2010.
[15] I. S. KIM. Wristwatch type health monitoring device capable of easily obtaining bio information [KR1020090099147], 2009.
[16] R. Kuperstein and Z. Sasson. Effects of Anti-hypertensive Therapy on Glucose and Insulin Metabolism and on Left Ventricular Mass. Circulation, 102:1802-1806, 2000.
[17] L. Arthur. Zhagafarovich and U. H. Bucarvich. Device and method for noninvasive measuring glucose level in the blood [U.S. Pat. No. 7,510,528], 2009.
[18] E. Matteucci and O. Giampietro. Circadian rhythm of blood pressure in diabetes mellitus: evidence, mechanisms and implications. Current diabetes reviews, 8(5):355-61, 2012.
[19] E. Matteucci and G. Ottavio. Ambulatory blood pressure monitoring and circadian rhythm of blood pressure in diabetes mellitus. EMJ Diabet., 1:38-43, 2013.
[20] P. M. Nilsson and R. Cifkova. Blood Pressure-Lowering Aspects of Lipid-Lowering and Anti-Diabetic Drugs. Pharmaceuticals, 4(1):1-6, 2010.
[21] Gianfranco Parati and Grzegorz Bilo. Should 24-h ambulatory blood pressure monitoring be done in every patient with diabetes? Diabetes care, 32 Suppl 2:S298-304, 2009.
[22] V. A. Rivas. Vital signals and glucose monitoring personal wireless system [U.S. Pat. No. 7,400,257], 2008.
[23] A. Porras, et al. Methods and systems for patient care [US20100137693], 2010.
[24] S. Senn. Cross-over Trials in Clinical Research. John Wiley & Sons, 2002.
[25] Y.-Y. Shen. Earphone-type physiological function detecting system [US20050177029], 2005.
[26] J. R. Taylor and K. M. Campbell. Home monitoring of glucose and blood pressure. American Family Physician, 76:255-260, 2007.
[27] B. E. Willner, et. al. Method and apparatus for using physical characteristic data collected from two or more subjects [U.S. Pat. No. 6,701,271], 2004.
[28] N. A. Zakopoulos, et. al. Diurnal correlation of ambulatory blood pressure and interstitial glucose in patients with normal glucose tolerance. Blood pressure monitoring, 13(6):309-17, 2008.
[29] L. A. Zhagafarovich and U. H. Bucarvich. Device and method for noninvasive measuring glucose level in the blood [US20060058596], 2006.
[30] L. Zhang, et. al. Blood pressure and blood sugar measuring instrument [CN101703395A], 2010.

It will thus be seen that further modifications and alternative embodiments of various aspects of the disclosure will be apparent to those skilled in the art in view of this description. Accordingly, this description is to be construed as illustrative only and is for the purpose of showing those skilled in the art the general manner of carrying out the invention. It is to be understood that the forms of the disclosure shown and described herein are to be taken as the presently preferred embodiments and shall be interpreted as illustrative and not in a limiting sense. Variations may be made in the models and methods described herein without departing from the spirit and scope of the invention as described in the following claims.

What is claimed:

1. A mixed model containing both fixed effects and random effects, comprising: q correlated response variables measured at the same time-point or at a matched time-point in a circadian rhythm between different days; multiple effects including p-level period, d-level day, t-level time-point within a day; g-level regimen; interactions of the said multiple effects; separable variance and covariance components of the q response variables at the different levels of between-period, between-day, between-time-point, and within-time-point; interactions among the effects; a statistical software tool to implement the mixed model; a computer to receive data from monitoring of the said response variables and to output the estimated responses, their differences and the statistical significance of the differences between regimens.

2. The statistical model of claim 1, wherein the q response variables have a variance-covariance matrix with no more than q(q+1)/2 different components representing their correlations at the same time-point or at a matched timepoint in a circadian rhythm between different days.

3. The statistical model of claim 1, wherein the q response variables have a variance-covariance matrix with no more than q(q+1)/2 different components representing their correlations between time-points on the same day.

4. The statistical model of claim 1, wherein the q response variables have a variance-covariance matrix with no more than q(q+1)/2 different components representing their correlations between days in the same period.

5. The statistical model of claim 1, wherein the q response variables have a variance-covariance matrix with no more than q(q+1)/2 different components representing their correlations between periods in the same sequence.

6. The statistical model of claim 1, wherein the q×q variance-covariance matrix in claim 2 and the q×q variance-covariance matrix in claim 3 form a new (q×t)×(q×t) variance-covariance matrix based on a compound symmetry structure, with the q×q variance-covariance matrix in claim 2 being repeated t times along the diagonal of the new matrix and the q×q variance-covariance matrix in claim 3 being repeated t×t times in the new matrix.

7. The statistical model of claim 1, wherein the (q×t)×(q×t) variance-covariance matrix in claim 6 and the q×q variance-covariance matrix in claim 4 form a new (q×t×d)×(q×t×d) variance-covariance matrix based on a compound symmetry structure, with the q×q variance-covariance matrix in claim 6 being repeated d times along the diagonal of the new matrix and the q×q variance-covariance matrix in claim 4 being repeated (t×d)×(t×d) times in the new matrix.

8. The statistical model of claim 1, wherein the (q×t×d)×(q×t×d) variance-covariance matrix in claim 7 and the q×q variance-covariance matrix in claim 5 form a new (q×t×d×p)×(q×t×d×p) variance-covariance matrix based on a compound symmetry structure, with the q×q variance-covariance matrix in claim 7 being repeated p times along the diagonal of the new matrix and the q×q variance-covariance matrix in claim 5 being repeated (t×d×p)×(t×d×p) times in the new matrix.

9. The statistical model of claim 1, wherein the (q×t×d×p)×(q×t×d×p) variance-covariance matrix in claim 8 represents the variance-covariance matrix of the (t×d×p) measurements of the q response variables.

10. The statistical model of claim 1, wherein the (q×t×d×p)×(q×t×d×p) variance-covariance matrix in claim 8 has no more than 2×q(q+1) components to estimate.

11. The statistical model of claim 1, wherein the output of the estimated responses, their differences, and the statistical significance of the differences is used to determine whether the g regimens are equivalent.

12. The statistical model of claim 1, wherein the output of the estimated responses, their differences, and the statistical significance of the differences is used to determine whether the q response variables respond equally to the regimens.

13. A mixed model containing both fixed effects and random effects, comprising: q correlated response variables measured at the same time-point or at a matched time-point in a circadian rhythm between different days; multiple effects including p-level period, d-level day, t-level time-point within a day; g-level regimen; interactions of the said multiple effects; separable variance and covariance components of the q response variables at the different levels of between-period, between-day, between-time-point, and within-time-point; interactions among the effects; a statistical software tool to implement the mixed model; a computer to receive data from monitoring of the said response variables and to output the estimated responses, their differences and the statistical significance of the differences between regimens.

14. The statistical model of claim 13, wherein one of the q response variables is the baseline reading of another response variable among that q response variables.

15. The statistical model of claim 13, wherein the number of days is one (d=1).

16. A statistical system comprising: correlated response variables measured at the same time-point or at a matched time-point in a circadian rhythm; synchronized sensors to provide continuous or discrete readings of the response variables; a receiver containing an antenna or antennas to receive, store, and transmit the readings; a computing device to receive the transmitted readings of the response variables measured at different levels of fixed and random effects that include regimen, period, day, time-point, and their interactions; a statistical software tool to implement a mixed model to process the received data and to output the estimated values, differences, statistical significance of changes, and the correlations of response variables.

17. The integrated receiver assembly of claim 16, wherein the electronics used to receive data from sensors is housed in an enclosure or on a mounting base not to exceed a weight limit, which is within ten ounces, preferably within six ounces, more preferably within four ounces, and most preferably within two ounces.

18. The integrated receiver assembly of claim 16, wherein the antennas used to receive data do not space apart further than eight inches, preferably within four inches, more preferably within two inches, and most preferably within one inch.

19. The response variables of claim 16 are monitored in a synchronized fashion so that their paired readings are taken at the same time-point or within an one-hour time window, preferably within a fifteen-minute time window, more preferably within a five-minute time window, and most preferably within a 30-second time window.

20. The mixed model of claim 16, comprising: q correlated response variables measured at the same time-point or at a matched time-point in a circadian rhythm between different days; multiple effects including p-level period, d-level day, t-level time-point within a day; g-level regimen; interactions of the said multiple effects; separable variance and covariance components of the q response variables at the different levels of between-period, between-day, between-time-point, and within-time-point; interactions among the effects; a statistical software tool to implement the mixed model; a computer to receive data from monitoring of the said response variables and to output the estimated responses, their differences and the statistical significance of the differences between regimens.

21. The statistical model of claim 20, wherein the q response variables have a variance-covariance matrix with no more than q(q+1)/2 different components representing their correlations at the same time-point or at a matched timepoint in a circadian rhythm between different days.

22. The statistical model of claim 20, wherein the q response variables have a variance-covariance matrix with no more than q(q+1)/2 different components representing their correlations between time-points on the same day.

23. The statistical model of claim 20, wherein the q response variables have a variance-covariance matrix with no more than q(q+1)/2 different components representing their correlations between days in the same period.

24. The statistical model of claim 20, wherein the q response variables have a variance-covariance matrix with no more than q(q+1)/2 different components representing their correlations between periods in the same sequence.

25. The statistical model of claim 20, wherein the q×q variance-covariance matrix in claim 21 and the q×q variance-covariance matrix in claim 22 form a new (q×t)×(q×t) variance-covariance matrix based on a compound symmetry structure, with the q×q variance-covariance matrix in claim 21 being repeated t times along the diagonal of the new matrix and the q×q variance-covariance matrix in claim 22 being repeated t×t times in the new matrix.

26. The statistical model of claim 20, wherein the (q×t)×(q×t) variance-covariance matrix in claim 25 and the q×q variance-covariance matrix in claim 23 form a new (q×t×d)×(q×t×d) variance-covariance matrix based on a compound symmetry structure, with the q×q variance-covariance matrix in claim 25 being repeated d times along the diagonal of the new matrix and the q×q variance-covariance matrix in claim 23 being repeated (t×d)×(t×d) times in the new matrix.

27. The statistical model of claim 20, wherein the (q×t×d)×(q×t×d) variance-covariance matrix in claim 26 and the q×q variance-covariance matrix in claim 24 form a new (q×t×d×p)×(q×t×d×p) variance-covariance matrix based on a compound symmetry structure, with the q×q variance-covariance matrix in claim 26 being repeated p times along the diagonal of the new matrix and the q×q variance-covariance matrix in claim 24 being repeated (t×d×p)×(t×d×p) times in the new matrix.

28. The statistical model of claim 20, wherein the (q×t×d×p)×(q×t×d×p) variance-covariance matrix in claim 27 represents the variance-covariance matrix of the (t×d×p) measurements of the q response variables.

29. The statistical model of claim 20, wherein the (q×t×d×p)×(q×t×d×p) variance-covariance matrix in claim 27 has no more than 2×q(q+1) components to estimate.

30. The statistical model of claim 20, wherein the output of the estimated responses, their differences, and the statistical significance of the differences is used to determine whether the g regimens are equivalent.

31. The statistical model of claim 20, wherein the output of the estimated responses, their differences, and the statistical significance of the differences is used to determine whether the q response variables respond equally to the regimens.

32. The mixed model of claim 16, comprising: q correlated response variables measured at the same time-point or at a matched time-point in a circadian rhythm between different days; multiple effects including p-level period, d-level day, t-level time-point within a day; g-level regimen; interactions of the said multiple effects; separable variance and covariance components of the q response variables at the different levels of between-period, between-day, between-time-point, and within-time-point; interactions among the effects; a statistical software tool to implement the mixed model; a computer to receive data from monitoring of the said response variables and to output the estimated responses, their differences and the statistical significance of the differences between regimens.

33. The statistical model of claim 32, wherein one of the q response variables is the baseline reading of another response variable among the q response variables.

34. The statistical model of claim 32, wherein the number of days is one (d=1).

35. The statistical model of claim 32, wherein the number of periods is one (p=1).
